(19) United States
(12) Patent Application Publication
(10) Pub. No.: US 2009/0186138 A1
(43) Pub. Date: Jul. 23, 2009

(54) Method of Flavoring

(75) Inventors: Xiaogen YANG, West Chester, OH (US); Yili WANG, Mason, OH (US); Robert G. EILERMAN, Manhasset, NY (US); Jason JORDAN, Cincinnati, OH (US)

Correspondence Address: CURATOLO SIDOTI CO., LPA, 24500 CENTER RIDGE ROAD, SUITE 280, CLEVELAND, OH, 44145, US

(73) Assignee: Givaudan SA, Vernier (CH)

(21) Appl. No.: 12/357,724

(22) Filed: Jan. 22, 2009

Related U.S. Application Data
(60) Provisional application No. 61/011,818, filed on Jan. 22, 2008.

(51) Int. Cl. A23L 1/22; C07C 69/67; C07D 307/20; C07D 309/32

(52) U.S. Cl. 426/442; 560/254; 560/193; 549/484; 549/417

(57) ABSTRACT

A method of providing a desirable vanilla beany flavor in an orally-receivable product, includes adding to a product base at least one of divanillyl succinate or at least one compound according to Formula I or Formula II

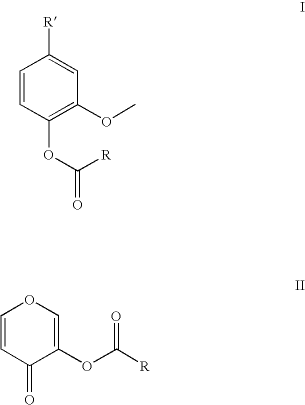

in which R′ is selected from the group consisting of hydrogen, methyl and —CHO, and R is selected from the group consisting of hydrogen. C1-C10 linear alkyl. C3-C10 branched alkyl, C2-C10 linear alkenyl, C3-C10 branched alkenyl, C1-C10 hydroxyalkyl, C2-C10 carboxyalkyl, and tetrahydrofuranyl.

This application claims the benefit of the filing date of U.S. Provisional Application for Patent Ser. No. 61/011,818, filed Jan. 22, 2008. incorporated herein by reference.

This disclosure relates to the achievement of vanilla beany flavor.

The achievement of a desirable vanilla beany flavor has long been a desire of flavorists. Hitherto it has not been possible to achieve by means other than using the natural material.

It has now been discovered that it is possible to provide such a desirable vanilla beany flavor. There is therefore provided a method of providing vanilla beany flavor in an orally-receivable product, comprising adding to a product base at least one of divanillyl succinate or at least one compound according to Formula I or Formula II

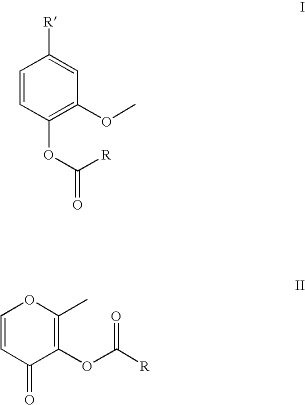

in which R′ is selected from the group consisting of hydrogen, methyl and —CHO, and R is selected from the group consisting of hydrogen. C1-C10 linear alkyl. C3-C10 branched alkyl, C2-C10 linear alkenyl, C3-C10 branched alkenyl, C1-C10 hydroxyalkyl, C2-C10 carboxyalkyl, and tetrahydrofuranyl.

Specific examples of R include hydrogen, methyl, ethyl, propyl, isopropyl, n-butyl, 1-methylpropyl, 2-methylpropyl, n-pentyl, 1-methylbutyl, 2-methylbutyl, 3-methylbutyl, 1-methylpentyl, 2-methylpentyl, 3-methylpentyl, 4-methylpentyl, 1-methylbut-2-ene, hydroxyethyl, carboxyethyl, and 2-tetrahydrofuranyl.

By “product base” is meant any combination of two or more materials that are combined with suitable flavor ingredients to produce a product that is orally-receivable, that is taken orally, either for ingesting (such as a foodstuff or a beverage) or for spitting (such as a mouthwash). All the standard materials known to the art may be used, and the skilled person will in every case be able to select suitable materials.

Some of the compounds of Formulae I and II above are novel materials. There is therefore also provided a compound selected from compounds according to Formulae III-VI, in which R has the value shown:

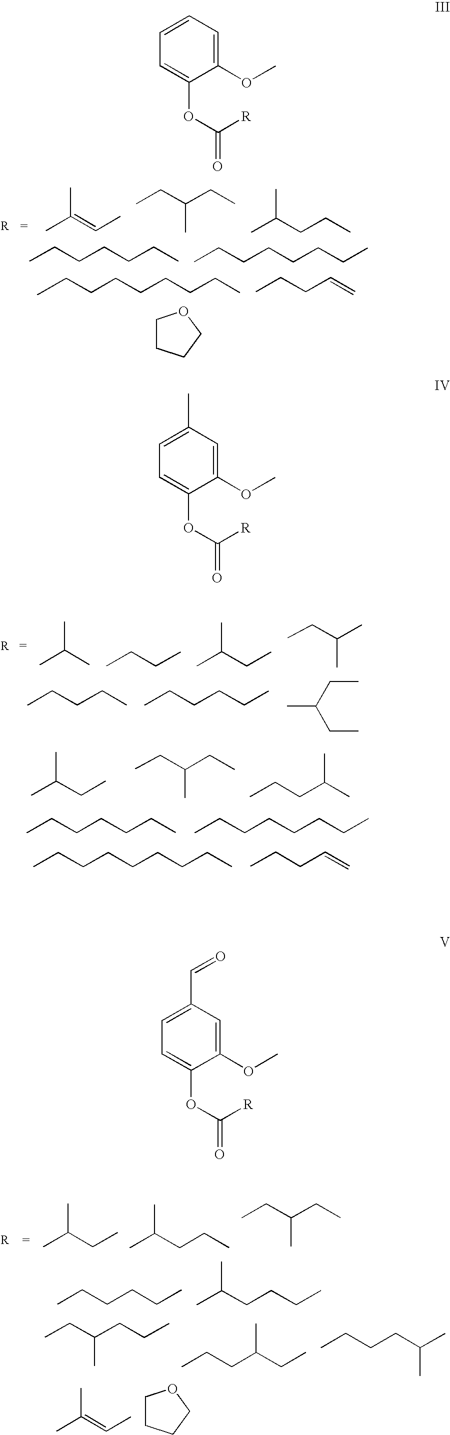

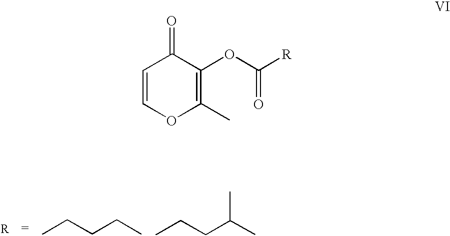

The compounds of Formula I may be prepared by standard methods known to the art, and the skilled person will be able to do so. For example, guaiacol and p-creosol esters may be prepared according to the methods described in the publication by Parish R. C. and Stock L. M. in J. Org. Chem., (1965) 30(3), 927-9, and vanillin and maltol esters may be prepared by the following methods:

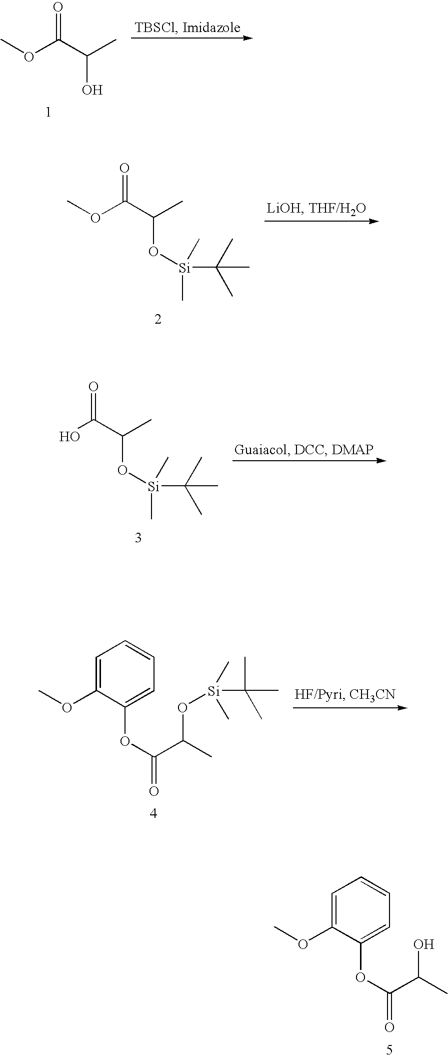

Particular examples of compounds that confer beany flavor are shown in the following tables. Table 1 contains particularly efficacious compounds, Table 2 less efficacious compounds and Table 3 still less efficacious compounds.

TABLE 1

| Common | | | |
|---|---|---|---|
| Name | Chemical Name | CAS | Structure |
| Guaiacol isovalerate | 2-methoxyphenyl 3- methylbutanoate | 68983-11-9 | 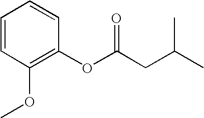 |
| Guaiacol propionate | 2-methoxyphenyl propionate | 7598-60-9 | 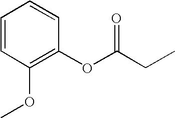 |
| Guaiacol 2- ethyl butyrate | 2-methoxyphenyl 2- ethylbutanoate | 723758-83-6 | 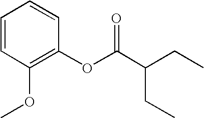 |
| Guaiacol butyrate | 2-methoxyphenyl butyrate | 4112-92-9 | 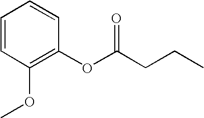 |
| Guaiacol 3- methyl pentanoate | 2-methoxyphenyl 3- methylpentanoate | No CAS | 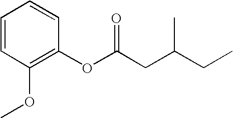 |
| Creosol propionate | 2-methoxy-4- methylphenyl propionate | 108439-89-0 | 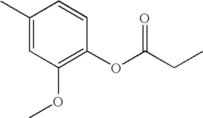 |
| Creosol butyrate | 2-methoxy-4- methylphenyl butyrate | No CAS | 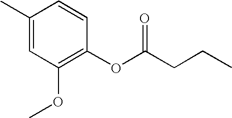 |
| Cresol valerate | 2-methoxy-4- methylphenyl valerate | No CAS | 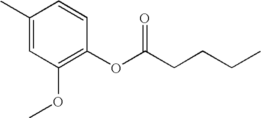 |
| Guaiacol succinate | 2-methoxyphenyl succinate | 39560-36-6 | 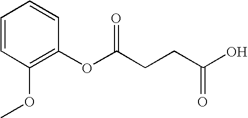 |
| Guaiacol lactate | 2-methoxyphenyl lactate | 59643-85-5 | 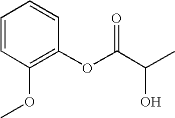 |
| Cresol acetate | 2-methoxy-4- methylphenyl acetate | 879-67-4 | 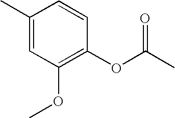 |
| Cresol 2- methyl butanoate | 2-methoxy-4- methylphenyl 2- methylbutanoate | No CAS | 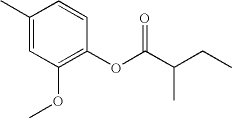 |
| Cresol 2- methyl pentanoate | 2-methoxy-4- methylphenyl 2- methylpentanoate | No CAS | 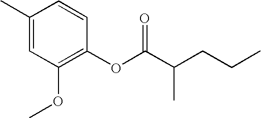 |
| Vanillin tetrahydro furan-2- carboxylate | 4-formyl-2- methoxyphenyl tetrahydrofuran-2- carboxylate | No CAS | 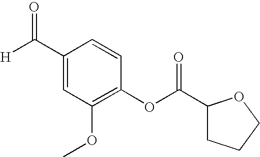 |
| Vanillin propionate | 4-formyl-2- methoxyphenyl propionate | 174143-90-9 | 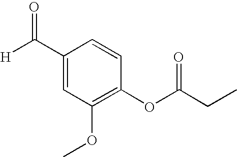 |
| Guaiacol tetrahydro furan-2- carboxylate | 2-methoxyphenyl tetrahydrofuran-2- carboxylate | No CAS | 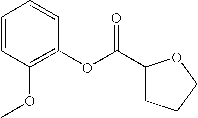 |
| Guaiacol valerate | 2-methoxyphenyl valerate | 531-39-5 | 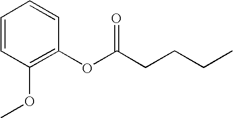 |
| Guaiacol hexanoate | 2-methoxyphenyl hexanoate | 613-00-3 | 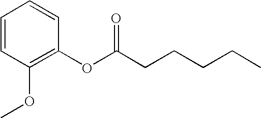 |
| Guaiacol octanoate | 2-methoxyphenyl octanoate | No CAS | 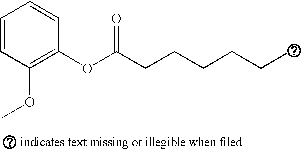 |
| Guaiacol nonanoate | 2-methoxyphenyl nonanoate | No CAS | 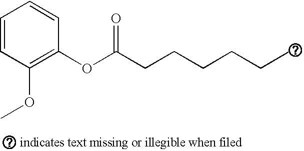 |
| Vanillin hexanoate | 4-formyl-2- methoxyphenyl hexanoate | 765298-67-7 | 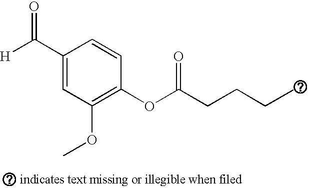 |
| Vanillin 4- methyl hexanoate | 4-formyl-2- methoxyphenyl 4- methylhexanoate | No CAS | 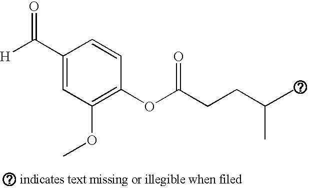 |
| Vanillin tiglate | 4-formyl-2- methoxyphenyl tiglate | No CAS | 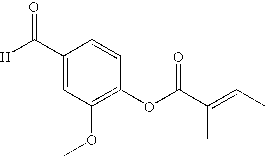 |

TABLE 2

| Common | Chemical | | |
|---|---|---|---|
| Name | Name | CAS | Structure |
| Guaiacol isobutytrate | 2-methoxyphenyl isobutyrate | 723759-62-4 | 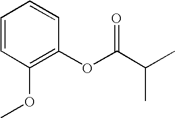 |
| Guaiacol 2- methyl butyrate | 2-methoxyphenyl 2-methylbutyrate | 379692-90-7 | 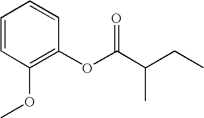 |
| Guaiacol tiglate | 2-methoxyphenyl tiglate | No CAS | 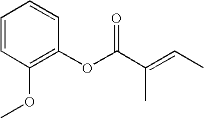 |
| Creosol hexanoate | 2-methoxy-4- methylphenyl hexanoate | No CAS | 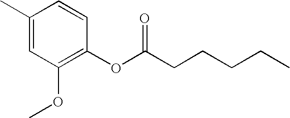 |
| Cresosol ethyl- 2-butyrate | 2-methoxy-4- methylphenyl ethyl-2-butyrate | No CAS | 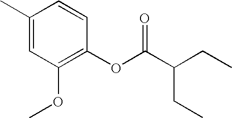 |
| Creosol 4- methyl pentanoate | 2-methoxy-4- methylphenyl 4- methylpentanoate | No CAS | 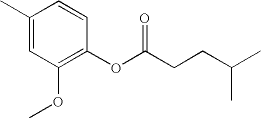 |
| Divanillyl succinate | bis(4-formyl-2- methoxyphenyl) succinate | 141186-17-6 | 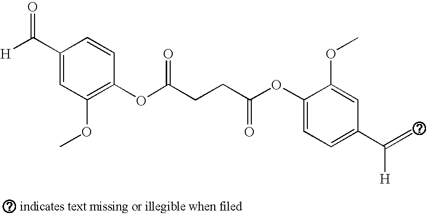 |
| Vanillin pentanoate | 4-formyl-2- methoxyphenyl pentanoate | 125261-96-3 | 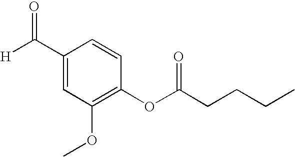 |
| Vanillin 5- methyl hexanoate | 4-formyl-2- methoxyphenyl 5-methylhexanoate | No CAS | 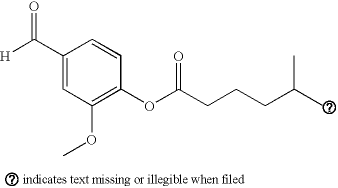 |
| Vanillin 2- methylbutyrate | 4-formyl-2- methoxyphenyl 2-methylbutyrate | No CAS | 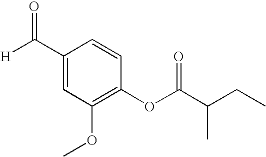 |
| Vanillin 2- methylvalerate | 4-formyl-2- methoxyphenyl 2-methylvalerate | No CAS | 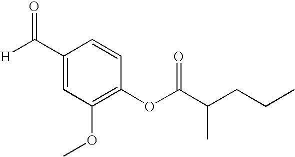 |

TABLE 3

| | Chemical | | |
|---|---|---|---|
| Common Name | Name | CAS | Structure |
| Creosol isobutyrate | 2-methoxy-4- methylphenyl isobutyrate | No CAS | 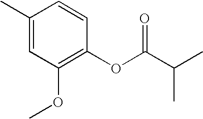 |
| Creosol isovalerate | 2-methoxy-4- methylphenyl isovalerate | No CAS | 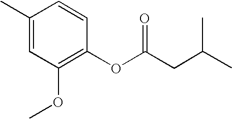 |
| Cresol 3-methyl pentanoate | 2-methoxy-4- methylphenyl 3-methyl pentanoate | No CAS | 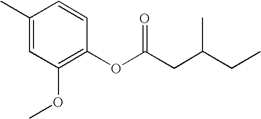 |
| Guaiacol 2- methyl pentanoate | 2-methoxyphenyl 2-methylpentanoate | No CAS | 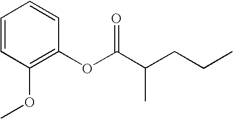 |
| Guaiacol isocaproate | 2-methoxyphenyl 4-methylpentanoate | 725736-75-4 | 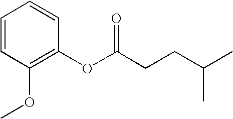 |
| Guaiacol heptanoate | 2-methoxyphenyl heptanoate | No CAS | 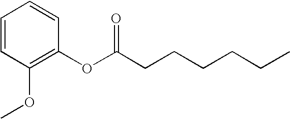 |
| Vanillin 3- methylbutanoate | 4-formyl-2- methoxyphenyl 3-methylbutanoate | 855938-06-6 | 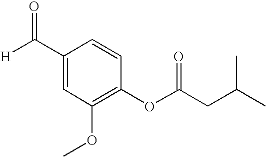 |
| Vanillin butyrate | 4-formyl-2- methoxyphenyl butyrate | 844634-13-5 | 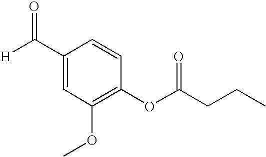 |

The compounds may be used in any orally-receivable product. There is therefore additionally provided a vanilla beany-flavored orally-receivable product comprising a product base in which is included at least one compound according to Formula I or Formula II, as hereinabove described.

Examples of such products include, but are not limited to, the following:

Beverages, including soft drinks and alcoholic beverages, tea, coffee and juices.

Sweet goods, including baked products such as cakes, cookies and biscuits, pies, cereals, cereal bars; confectionery, such as hard candy, chewing gum, chocolates, soft candies and jellies.

Dairy products, including milk, ice cream and yogurt.

Savory products, including snack foods, dressings, dips, potato chip snacks.

Medicinal products, such as syrups, chewable tablets.

Dentifrices, including toothpastes, tooth powders, tooth gels, mouthwashes and edible films.

The method will now be further described with reference to the following examples. These describe particular embodiments and are not intended to be in any way limiting.

### EXAMPLE 1

### Preparation of Guaiacol Lactate

To a solution of lactic acid methyl ester 1 (25 g, 9.6 mmol) and DMF (50 ml, 2 ml/g solvent) was added tert-butyldimethylsilyl chloride (43.4 g, 288.2 mmol), followed by imidazole (40.8 g, 144.2 mmol). The reaction was stirred at room temperature overnight and then quenched with water. The mixture was extracted with ethyl acetate (3×50 ml). The combined organic layers were washed with 1 N HCl, water and saturated NaCl, dried (Na₂SO₄), filtered, and concentrated. The crude was purified by flash column chromatography eluting with 100% hexane to obtain 41.8 g (80%) of product (“Product 2”).

To a mixture of Product 2 (3 g, 13.8 mmol) and THF (130 ml) at 0° C. was added 1.13 g (27.5 mmol) of LiOH in 130 ml water. After being stirred at RT for about 4 h, the THF was removed under vacuum. The resulting aqueous mixture was washed with EtOAc (2×50 ml). The EtOAc layers were extracted with saturated NaHCO₃. The aqueous layers were combined, acidified to pH=5 with saturated KHSO₄, and then extracted with EtOAc. The resulting organic layers were combined, dried and concentrated to afford 2.49 g (89%) of product as clear oil (“Product 3”). Product 3 may be used without further purification.

To a solution of Product 3 (2.49 g, 12.2 mmol) in 50 ml CH₂Cl₂ was treated DCC at 0° C. After the mixture was stirred for 10 min, guaiacol (2.3 g, 18.5 mmol) and DMAP (0.15 g, 1.2 mmol) were added to the reaction mixture at 0° C. The reaction was stirred at room temperature for over night. More methylene chloride was added to the reaction mixture. The reaction mixture was then washed with 1N HCl and water, dried over Na₂SO₄ and concentrated in vacuo. The crude product was purified on silica with 5% EtOAc/Hex and gave 1.74 g (46%) of product (“Product 4”).

Product 4 (1.74 g, 5.6 mmol) was dissolved in 20 ml CH₃CN, cooled to 0° C. HF/Pyr (2 ml) was added, then the ice bath was removed and the reaction was stirred at room temperature for 2.5 hrs. Water was added into the reaction mixture and extracted back with EtOAc (3×50 ml), washed the organic layers with water, brine, dried and concentrated. Flash chromatography (20% EtOAc/Hex) yielded 0.93 g (86%) of final product as clear oil.

### EXAMPLE 2

### Preparation of Guaiacol Succinate

Guaiacol (2.23 g, 17.9 mmol), succinic anhydride (2.7 g, 26.9 mmol), and 4-(dimethylamino)pyridine (50 mg, 0.40 mmol) were dissolved in 10 ml pyridine and left for 2 days at room temperature. After addition of 50 ml of CH₂Cl₂, the resulting solution was washed with 1 N HCl (3×50 ml), water, brine, dried (Na₂SO₄), and concentrated. Flash chromatography (20% EtOAc/Hex to 100% EtOAc) on silica gel yielded 1.47 g (37%) of white solid as final product.

### EXAMPLE 3

### Preparation of 2-Methoxyphenyl tetrahydrofuran-2-carboxylate

2-Tetrahydrofuroic acid (16 g, 137.8 mmol) was dissolved in 70 ml CH₂Cl₂ and was treated with 200 ml of 2 M solution of oxalyl chloride in CH₂Cl₂ and 0.2 ml of DMF at room temperature. The reaction was stirred at room temperature for 2.5 h. An excess amount of oxalyl chloride was removed under vacuo. To a solution of guaiacol (5 g, 40.3 mmol) in 50 mL CH₂Cl₂ was added freshly prepared tetrahydrofuran-2-carbonyl chloride (8.1 g, 60.4 mmol) and 6.6 ml (100.6 mmol) of pyridine at room temperature. The resulting mixture was stirred at room temperature for 15 min, quenched with MeOH. The reaction mixture was poured into 50 mL 1 N HCl. The aqueous layer was back-extracted with CH₂Cl₂ (3×50 mL), and the organic layers were combined and washed with 1 N HCl, water, saturated NaCl, dried (Na₂SO₄) and concentrated. Purification via flash chromatography on SiO₂ (20% EtOAc/Hex) gave 8.95 g (63%) of product.

### EXAMPLE 4

### Preparation of Vanillin Propionate

To a solution of vanillin (2.75 g, 18.1 mmol) in CH₂Cl₂ (30 mL) at room temperature was added propyl chloride (3.7 mL, 27.1 mmol) and pyridine (3.7 mL, 45.2 mmol). The resulting mixture was stirred overnight. The reaction mixture was poured into 1 N HCl, and the layer was separated. The aqueous layer was extracted with CH₂Cl₂ (3×50 mL), and the organic layers were combined. The combined CH₂Cl₂ extracts were washed with H₂O, saturated NaCl, dried with Na₂SO₄, concentrated, and purified by flash chromatography (20% EtOAc/Hex) to afford 2.06 g (55%) of vanillin propionate.

### EXAMPLE 5

### Preparation of 4-formyl-2-methoxyphenyl tetrahydofuran-2-carboxylate (vanillin 2-tetrahydrofuroate)

2-Tetrahydrofuroic acid (16 g, 137.8 mmol) was dissolved in 70 mL CH₂Cl₂ and was treated with 200 mL of 2M solution of oxalyl chloride in CH₂Cl₂ and 0.2 mL of DMF at room temperature. The reaction was stirred at room temperature for 2.5 h. An excess amount of oxalyl chloride was removed under vacuo. To a solution of vanillin (5 g, 32.9 mmol) in 50 mL CH₂Cl₂ was added freshly prepared tetrahydrofuran-2-carbonyl chloride (6.6 g, 49.5 mmol) and 7 mL (82.2 mmol) of pyridine at room temperature. The resulting mixture was stirred at room temperature for 30 min, then reflux for 2 h and quenched with MeOH at room temperature. Poured the reaction mixture to 1 N HCl (50 mL). The aqueous layer was extracted with CH₂Cl₂ (3×50 mL) and the organic layers combined, washed with 1 N HCl, H₂O, saturated NaCl and dried (Na₂SO₄). The solvent was removed in vacuo and the resulting oil was purified by chromatography on SiO₂ to provide 3.2 g (39%) of desired product.

### EXAMPLE 6

### Alcoholic Beverage with Vanilla Flavor

The compounds listed in Table 4 were added to the following alcoholic beverage base at a concentration of 0.5 or 1 ppm.

| Ingredient | Amount | |
|---|---|---|
| Water | 852.78 | mL |
| Sodium Benzoate | 0.25 | g |
| Potassium Sorbate | 0.15 | g |
| Sodium Citrate | 0.3 | g |
| Ethanol 190 proof | 52.6 | mL |
| Vanilla flavor V1* (5%) | 0.1 | g |
| High Fructose Corn Syrup-55 | 95 | mL |

*commercially-available vanilla flavor ex Givaudan Flavors Corp.

The flavor profile was evaluated by three vanilla flavorists. Comparing the resulting composition with the aroma and taste profile of the starting beverage, the vanilla flavor was found to be enhanced and/or improved. The enhancing effects are listed in Table 4.

TABLE 4

| Compound | Flavor Profile Description in the Beverage Base |
|---|---|
| Creosol Acetate | finish is a little more brown and fatty, slightly smoky, |
| | slightly more vanillin, improvement is a little subtle |
| Vanillin | enhances brown notes and makes profile a little more |
| tetrahydrofuran-2- | |
| carboxylate | resinous |
| Vanillin | fatty, oily, gives the impression of high pressure |
| propionate | extraction. |
| Guaiacol | more smoky and dark, interesting, |
| tetrahydrofuran-2- | |
| carboxylate | |
| Guaiacol valerate | beany, fruity finish, added guaiacol-like aroma, but |
| | sweeter |
| Guaiacol | helps aroma, taste is a little more brown, resinous and |
| octanoate | slightly phenolic |
| Vanillin tiglate | smoothes, balances, beany, sweet vanillin-like, |
| | improves the overall flavor |

### EXAMPLE 7

### Cola Beverage with Vanilla Flavor

The compounds listed in Table 5 were added to the following cola beverage base at a concentration of 0.5 or 1 ppm.

| Ingredient | Amount | |
|---|---|---|
| Cola Syrup Base | 167 | g |
| Vanilla Flavor V2* (5%) | 0.01 | g |
| Carbonated Water (Schweppes) | 833 | g |

*commercially-available vanilla flavor ex Givaudan Flavors Corp.

The flavor profile was evaluated by three vanilla flavorists. Comparing the resulting composition with the aroma and taste profile of the starting beverage, the vanilla flavor was enhanced and/or improved. The enhancing effects are listed in Table 5:

TABLE 5

| | Amount | Flavor Profile Description in the Cola |
|---|---|---|
| Compound | (ppm) | Base |
| Creosol Acetate | 0.5 | slightly sweeter ending, beany, phenolic, |
| | | slightly sweeter |
| Vanillin | 0.5 | Smoother taste, sweeter |
| tetrahydrofuran- | 1 | same as 0.5 ppm, gives middle |
| 2-carboxylate | | smoothness, beany |
| Vanillin propionate | 0.5 | nice beany, slightly smoky, slightly nutty |
| Guaiacol | 0.5 | very slightly smoky, slightly brown |
| tetrahydrofuran- | 1 | beany, phenolic, slightly medicinal |
| 2-carboxylate | | |
| Guaiacol valerate | 0.5 | beany, phenolic, medicinal, a little more |
| | 1 | brown note, some sweet character at the |
| | | end, nondescript nice background note |
| Guaiacol octanoate | 0.5 | Slightly sweetening, very slightly |
| | | fatty/resinous |
| Vanillin tiglate | 0.5 | slightly sweetening |
| | 1 | smoothed out the imbalance in the cola |

### EXAMPLE 8

### Cookie with Vanilla Flavor

Compounds listed in Table 6 were added to the following cookie base at a concentration of 200 ppm.

| Ingredient | Amount |
|---|---|
| Cookie Base | 998 g |
| Vanilla Flavor V1 (5%) | 2 g |

The flavor profile was evaluated by three vanilla flavorists. Comparing the resulting composition with the aroma and taste profile of the cookie base, the vanilla flavor was enhanced and/or improved. The enhancing effects are listed in Table 6.

TABLE 6

| Compound | Flavor Profile Description in the Cookie Base |
|---|---|
| Creosol Acetate | Beany, resin, fatty, creamy, vanilla-like, sweeter |
| Vanillin | Creamier, baked, vanillin-like, positive for overall |
| tetrahydrofuran- | character |
| 2-carboxylate | |
| Guaiacol | Beany, smoky, resinous, phenolic/guaiacol-like |
| tetrahydrofuran- | |
| 2-carboxylate | |
| Vanillin propionate | Smoky, vanilla character, brown |
| Guaiacol valerate | Better beany character, fruity |
| Guaiacol octanoate | Mild vanilla, smoothes flavor |
| Vanillin tiglate | Beany, brown baked notes, creamy, doughy |

### EXAMPLE 9

### Chewing Gum with Vanilla Flavor

The compounds listed in Table 7 were added to the following chewing gum base at a concentration of 250 ppm.

| Ingredient | Amount |
|---|---|
| Gum Base | 995 g |
| Vanilla Flavor V1 (5%) | 5 g |

The flavor profile was evaluated by three vanilla flavorists. Comparing the resulting composition with the aroma and taste profile of the chewing gum base, the vanilla flavor was enhanced and/or improved. The enhancing effects are listed in Table 7.

TABLE 7

| Compound | Flavor Profile Description in the Gum Base |
|---|---|
| Creosol Acetate | Slightly brown, more sweet, caramel or |
| | cocoa like, good flavor |
| Vanillin tetrahydrofuran-2- | Resinous, beany, good enhancement of |
| carboxylate | flavor |
| Guaiacol tetrahydrofuran-2- | Nice beany, brown, slightly fatty, resinous |
| carboxylate | |
| Vanillin propionate | Beany, resinous, brown, sweet |
| Guaiacol octanoate | More beany, brown, resinous, sweet |

### EXAMPLE 10

### Milk with Vanilla Flavor

The compounds listed in Table 8 were added to the following milk base at a concentration of 20 ppm.

| Ingredient | Amount |
|---|---|
| Whole Milk | 908.82 g |
| Sucrose | 90.88 g |
| Vanilla Beany Flavor V1 (5%) | 0.30 g |

The flavor profile was evaluated by three vanilla flavorists. Comparing the resulting composition with the aroma and taste profile of the milk base, the vanilla flavor was enhanced and/or improved. The enhancing effects are listed in Table 8.

TABLE 8

| Compound | Flavor Profile Description in the Milk Base |
|---|---|
| Creosol Acetate | sweeter, moot on guaiacol, rummy, smoky, beany |
| Vanillin | slightly smoky, brown, enhances beany notes, |
| tetrahydrofuran-2- | sweeter, moot on guaiacol |
| carboxylate | |
| Vanillin propionate | increased dried fruit, creamy, caramel or toffee- |
| | like, rummy finish |
| Guaiacol valerate | softens smoky notes, slightly caramel-like, beany, |
| | dried fruit/raisin, nice |
| Guaiacol octanoate | beany, woody, slightly sweeter, a little fatty, |
| | resinous, subtle improvement |
| Vanillin tiglate | rummy, alcoholic, some increased beany/seedy |
| | notes, slightly sweeter |

### EXAMPLE 11

### Yogurt with Vanilla Flavour

The compounds listed in Table 9 were added to the following yogurt base at a concentration of 20 ppm.

| Ingredient | Amount |
|---|---|
| Plain Yogurt | 908.82 g |
| Sucrose | 90.88 g |
| Vanilla Flavor V2 (5%) | 0.3 g |

The flavor profile was evaluated by three vanilla flavorists. Comparing the resulting composition with the aroma and taste profile of the yogurt base, the vanilla flavor was enhanced and/or improved. The enhancing effects are listed in Table 9.

TABLE 9

| Compound | Flavor Profile Description in the Yogurt Base |
|---|---|
| Creosol Acetate | Sweeter, beany, nice, covers some of the phenolic |
| | notes of reference |
| Vanillin | Beany, browner, slightly smoky |
| tetrahydrofuran- | |
| 2-carboxylate | |
| Vanillin propionate | Beany, brown, almost chocolate, suppresses |
| | phenolic notes of reference. Slightly caramelic, |
| | raisin, increased sweetness |
| Guaiacol | Beany, smoky, brown, slightly caramelic, raisin, |
| tetrahydrofuran-2- | sweet |
| carboxylate | |
| Guaiacol valerate | Very smoky, rummy, beany, slightly resinous, |
| | slightly more brown |
| Guaiacol octanoate | Finish has nice beany note, sweeter, dried fruit |
| Vanillin tiglate | Brown, vanillin, beany-like, slight sweet rum and |
| | raisin note |

### EXAMPLE 12

### Cough Syrup with Vanilla Flavor

The compounds listed in Table 10 were added to the following cough syrup base at a concentration of 100 ppm.

| Ingredient | Amount | |
|---|---|---|
| Cough Syrup | 7.49 | g |
| Water | 991.51 | g |
| Vanilla Flavor V1 (5%) | 1 | g |

The flavor profile was evaluated by three vanilla flavorists. Comparing the resulting composition with the aroma and taste profile of the cough syrup base, the vanilla flavor was enhanced and/or improved. The enhancing effects are listed in Table 10.

TABLE 10

| | Flavor Profile Description in the |
|---|---|
| Compound | Cough Syrup Base |
| Vanillin tetrahydrofuran-2- | Sweet, brown, balances overall profile |
| carboxylate | |
| Vanillin propionate | Resinous, fatty, beany, rummy finish |

### EXAMPLE 13

### Mouthwash with Vanilla Flavor

The compounds listed in Table 11 were added to the following mouthwash base at a concentration of 50 ppm.

| Ingredient | Amount | |
|---|---|---|
| Mint flavored mouthwash | 19.98 | g |
| Water | 979.02 | g |
| Vanilla Flavor V1 (5%) | 1 | g |

The flavor profile was evaluated by three vanilla flavorists. Comparing the resulting composition with the aroma and taste profile of the mouthwash base, the vanilla flavor was enhanced and/or improved. The enhancing effects are listed in Table 11.

TABLE 11

| Compound | Flavor Profile Description in the Mouthwash Base |
|---|---|
| Creosol Acetate | A little more brown, sweeter, improved beany |
| Vanillin | Nice beany, sweet, resinous, slightly rummy, sweet |
| tetrahydrofuran- | estery |
| 2-carboxylate | |
| Vanillin propionate | Beany improvement, resinous, slightly rummy |
| Guaiacol octanoate | Beany, slightly sweet, doughy, balanced |
| Vanillin tiglate | Fatty mouthfeel |

It will be understood that the embodiments described herein are merely exemplary and that variations and modifications can be made by one skilled in the art without departing from the spirit and the scope of the invention. It should be understood that the embodiments described above are not only in the alternative, but can be combined.

We claim:

1. A method of providing vanilla beany flavor in an orally-receivable product, comprising adding to a product base at least one of divanillyl succinate or at least one compound according to Formula I or Formula II 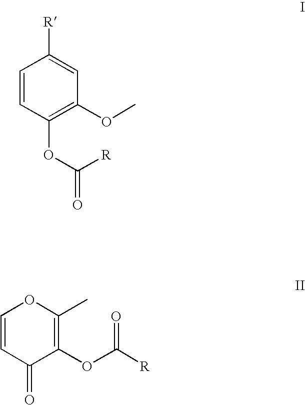
in which R′ is selected from the group consisting of hydrogen, methyl and —CHO, and R is selected from the group consisting of hydrogen, C1-C10 linear alkyl, C3-C10 branched alkyl, C2-C10 linear alkenyl, C3-C10 branched alkenyl, C1-C10 hydroxyalkyl, C2-C10 carboxyalkyl, and tetrahydrofuranyl.

2. The method according to claim 1, in which R is selected from the group consisting of hydrogen, methyl, ethyl, propyl, isopropyl, n-butyl, 1-methylpropyl, 2-methylpropyl, n-pentyl, 1-methylbutyl, 2-methylbutyl, 3-methylbutyl, 1-methylpentyl, 2-methylpentyl, 3-methylpentyl, 4-methylpentyl, 1-methylbut-2-ene, hydroxyethyl, carboxyethyl, and 2-tetrahydrofuranyl.

3. A compound of Formula III 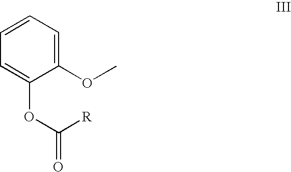
in which R is a moiety selected from the group consisting of

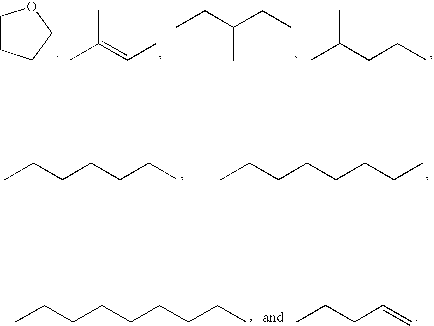

4. A compound of Formula IV 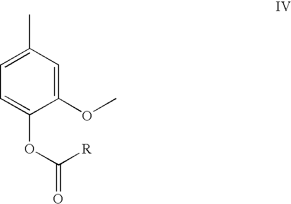
in which R is a moiety selected from the group consisting of

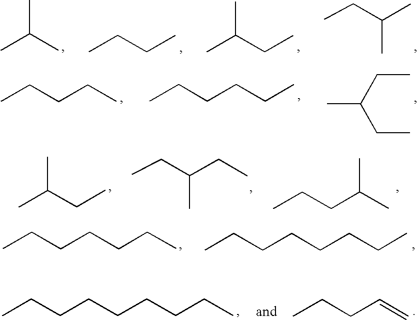

5. A compound of Formula V 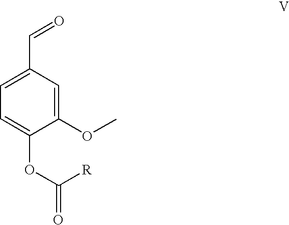
in which R is a moiety selected from the group consisting of

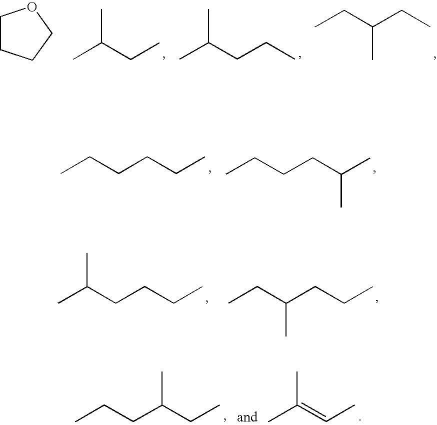

6. A compound of Formula VI 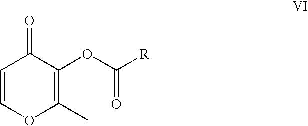
in which R is a moiety selected from the group consisting of

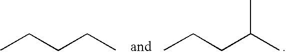

7. A vanilla beany-flavored orally-receivable product comprising a product base in which is included at least one of divanillyl succinate or at least one compound according to Formula I or Formula II 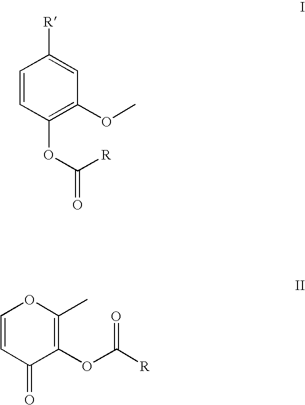
in which R′ is selected from the group consisting of hydrogen, methyl and —CHO, and R is selected from the group consisting of hydrogen, C1-C10 linear alkyl, C3-C10 branched alkyl, C2-C10 linear alkenyl, C3-C10 branched alkenyl, C1-C10 hydroxyalkyl, C2-C10 carboxyalkyl, and tetrahydrofuranyl.

8. The product according to claim 7, in which R is selected from the group consisting of hydrogen, methyl, ethyl, propyl, isopropyl, n-butyl, 1-methylpropyl, 2-methylpropyl, n-pentyl, 1-methylbutyl, 2-methylbutyl, 3-methylbutyl, 1-methylpentyl, 2-methylpentyl, 3-methylpentyl, 4-methylpentyl, 1-methylbut-2-ene, hydroxyethyl, carboxyethyl, and 2-tetrahydrofuranyl.

9. The product according to claim 7, comprising at least one of beverages, sweet goods, dairy products, savory products, medicinal products, or dentifrices.
